(19) United States
(12) Patent Application Publication
(10) Pub. No.: US 2019/0101540 A1
(43) Pub. Date: Apr. 4, 2019

(54) Angiogenesis Biomarkers Associated With Disease Progression in Lung Cancer

(71) Applicant: Rush University Medical Center, Chicago, IL (US)

(72) Inventor: Jeffrey A. Borgia, Chicago, IL (US)

(73) Assignee: Rush University Medical Center, Chicago, IL (US)

(21) Appl. No.: 16/152,896

(22) Filed: Oct. 5, 2018

Related U.S. Application Data
(62) Division of application No. 15/111,388, filed on Jul. 13, 2016, now abandoned.
(60) Provisional application No. 61/927,076, filed on Jan. 14, 2014.

(51) Int. Cl. G01N 33/574; C12Q 1/6886

(52) U.S. Cl. CPC G01N 33/57423; C12Q 2600/158; C12Q 2600/112; C12Q 1/6886

(57) ABSTRACT

Methods and kits are provided for determining lung cancer disease progression or for pathological stratification of solitary indeterminate nodules detected during lung cancer screening or for determining the likelihood of disease progression in a subject. The method includes obtaining a biological sample from the subject and assaying a level in the biological sample of a biomarker in a panel of biomarkers where the panel includes at least one biomarker. The method further includes comparing the biomarker level in the subject's sample to a cutoff value for each biomarker measured for the panel of biomarkers and determining whether the biomarker level is above or below the cutoff value.

### RELATED APPLICATIONS

This application is a division of application Ser. No. 15/111,388, filed Jul. 13, 2016, which claims the benefit under 35 U.S.C. § 371 of International Application No. PCT/US2015/011196, filed Jan. 13, 2015, which claims the benefit of U.S. Provisional Application No. 61/927,076, filed Jan. 14, 2014, which are incorporated by reference herein in their entirety.

### TECHNICAL FIELD

The present invention relates to methods and kits for identifying patients with different stages of lung cancer progression, for pathological stratification of solitary indeterminate nodules detected during lung cancer screening, for prognosticating patients likely to have disease recurrence and for aiding in determining treatments for patients with disease and for treatment monitoring.

### BACKGROUND

Mechanistic insights into tumor progression in lung cancer and a range of other carcinomas have advanced considerably over the last decade. The dysregulation of angiogenesis and lymphangiogenesis associated with disease progression have been perhaps the most studied in this mechanistic realm. Whereas normal vasculature is static, developing tumors require new vasculature to support their growth which is largely unstable (1). Although angiogenesis was initially thought to be a process limited to the later stage of development for large, rapidly growing tumors, more recently investigators have demonstrated signals for angiogenesis are unregulated early in tumorgenesis. This angiogenesis ‘switch’ is turned ‘on’ even at the level of dysplasia's and carcinoma in situ (2, 3). Angiogenesis is governed by a complex balance between pro- and anti-angiogenesis factors, the best described include vascular endothelial growth factor-A (VEGF-A) and thrombospondin-1 (TSP-1), respectively (1). Efforts are now ongoing to better identify biomarkers of angiogenesis in NSCLC, particularly concerning the differential temporal expression of these with disease progression (4).

The overall aim of this study was to globally evaluate angiogenesis biomarkers across a range of distinct, clinically-relevant phases of lung adenocarcinoma presentation and progression. This spectrum includes: individuals being screened for lung cancer and found to either have no detectable nodules or a positive CT scan (with two-years of no malignant behavior); stage I disease; cases of locally-advanced (N+) disease, and those with disseminated disease. One objective of the study was to evaluate the ability of circulating angiogenesis biomarkers to risk stratify indeterminate solitary nodules detected via low-dose CT screening protocols and, amongst the malignant cases, to determine which cases of stage I disease are at highest risk of disease recurrence. Another objective was to evaluate the association of angiogenesis biomarkers with both progression-free survival (PFS) and overall survival (OS) in each cohort of lung adenocarcinoma.

There is a need in the art for screening methods and kits that distinguish molecular signatures of benign versus malignant nodules for risk stratification of patients with indeterminate nodules and that identify patients that are at the highest risk of disease recurrence. There is also a need in the art for methods and kits for identifying patients with different stages of lung cancer progression and for aiding in the determining of optimal treatment plan for patients with advanced disease, and for treatment monitoring.

### BRIEF SUMMARY

Methods and kits are provided for determining lung cancer disease progression or for pathological stratification of solitary indeterminate nodules detected during lung cancer screening or for determining the likelihood of disease progression in a subject. The method incudes obtaining a biological sample from the subject and assaying a level in the biological sample of a biomarker in a panel of biomarkers where the panel includes at least one biomarker. The method further includes comparing the biomarker level in the subject's sample to a cutoff value for each biomarker measured for the panel of biomarkers and determining whether the biomarker level is above or below the cutoff value.

### BRIEF DESCRIPTION OF THE DRAWINGS

FIGS. 1A-1D illustrate representative ‘Box and Whisker’ plots indicating distributions of angiogenesis biomarkers across the patient groups. Distributions of IL-8 (FIG. 1A), HGF (FIG. 1B), VEGF-C (FIG. 1C), and VEGF-D (FIG. 1D) for the following groups: high-risk for lung cancer and negative LDCT imaging (‘Control’; n=20); High-risk for lung cancer with detected solitary pulmonary nodules (‘Benign Nodule’; n=36); Stage I lung adenocarcinoma (‘Stage I’; n=75); Locally-advanced (N+) disease (‘Locally Advanced’; n=60); and Stage IV disease (‘Stage IV’; n=68). Significance via Mann-Whitney Rank Sum tests is indicated as 1 (p≤0.05), 2 (p≤0.01), and 3 (p≤0.001), with actual values provided in Table I. (Note: not all extreme and outlier values are displayed to permit appropriate scaling of plots.)

FIGS. 2A-2D show Kaplan-Meier survival curves illustrating clinical outcomes associated with selected biomarkers within the stage I cohort. Kaplan-Meier curves shown for progression-free survival (PFS) for VEGF-C (FIG. 2A) and VEGF-D (FIG. 2B) or overall survival (OS) with VEGF-D (FIG. 2C) and VEGF-A (FIG. 2D). Cutoff values and number of cases in each arm are indicated as well as the log-rank p-value.

FIGS. 3A-3D illustrate representative ‘Box and Whisker’ plots indicating distributions of angiogenesis biomarkers relevant to lung adenocarcinoma detection (FIGS. 3A and 3B) and locoregional progression (FIGS. 3C and 3D). Distribution of G-CSF (FIG. 3A) and VEGF-D (FIG. 3B) are shown to illustrate differences in biomarker levels relevant to assigning pathological significance to indeterminate nodules found during a CT scan. For this, groups presented include the ‘benign nodule’ group (n=36) and the stage I adenocarcinoma (n=75) group. Also, EGF (FIG. 3C) and HGF (FIG. 3D) are shown for their relevance to locoregional progression. For this, cases of stage I lung adenocarcinoma (n=75) and locally-advanced (N+) disease (n=60) were included. Significance via Mann-Whitney Rank Sum tests is indicated in each panel with actual values provided in Table I.

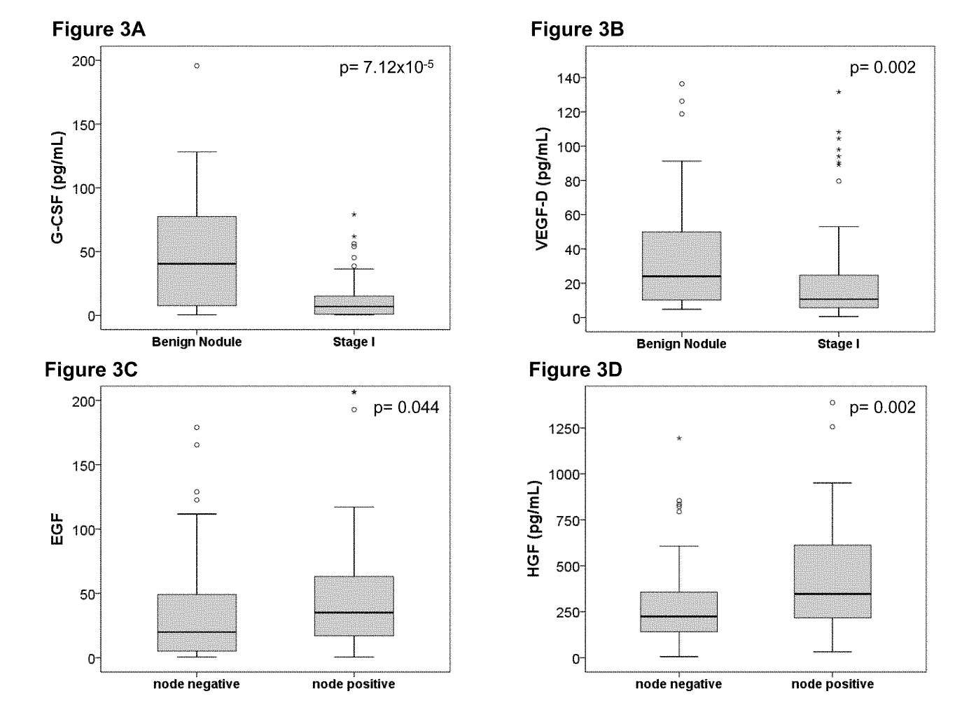

FIGS. 4A-4D show Kaplan-Meier survival curves illustrating clinical outcomes associated with selected biomarkers within the stage IV cohort. Kaplan-Meier curves shown for progression-free survival (PFS) for endothelin-1 (FIG. 4A) and Leptin (FIG. 4B) or overall survival (OS) with angiopoeitin-2 (FIG. 4C) and endothelin-1 (FIG. 4D). Cutoff values and number of cases in each arm are indicated as well as the log-rank p-value.

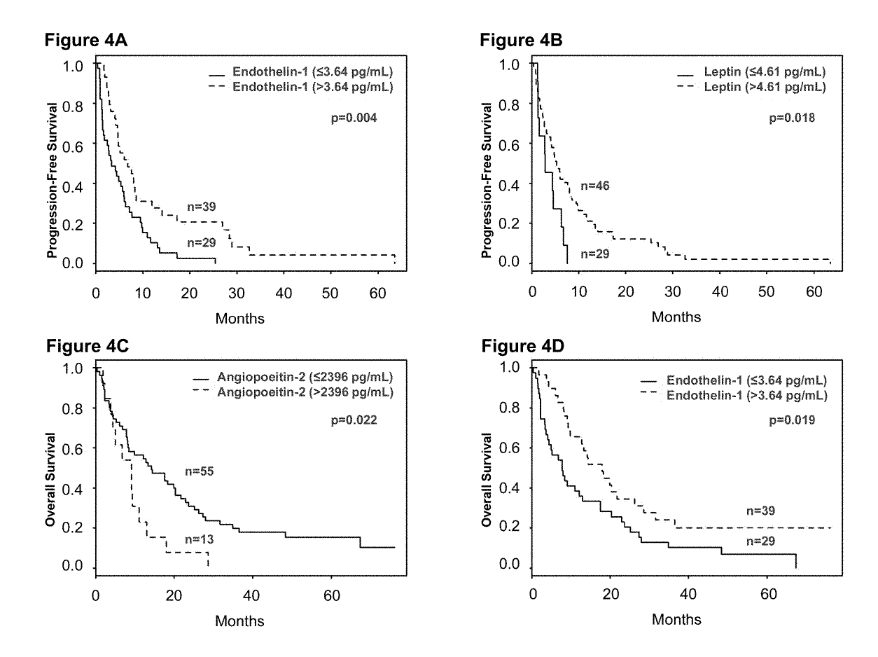

### DETAILED DESCRIPTION

The present invention will utilize one or more biomarkers in a panel of biomarkers measured in a biological sample obtained from a subject to determining lung cancer disease progression or for pathological stratification of solitary indeterminate nodules detected during lung cancer screening or for determining the likelihood of disease progression in a subject.

The term “biomarker” as used herein, refers to any biological compound that can be measured as an indicator of the physiological status of a biological system. A biomarker may comprise an amino acid sequence, a nucleic acid sequence and fragments thereof. Exemplary biomarkers include, but are not limited to cytokines, chemokines, growth and angiogenic factors, metastasis related molecules, cancer antigens, apoptosis related proteins, proteases, adhesion molecules, cell signaling molecules and hormones.

“Measuring” or “measurement” means assessing the presence, absence, quantity or amount (which can be an effective amount) of a given substance within a sample, including the derivation of qualitative or quantitative concentration levels of such substances, or otherwise evaluating the values or categorization of a subject's clinical parameters. Alternatively, the term “detecting” or “detection” may be used and is understood to cover all measuring or measurement as described herein.

The terms “sample” or “biological sample” as used herein, refers to a sample of biological fluid, tissue, or cells, in a healthy and/or pathological state obtained from a subject. Such samples include, but are not limited to, blood, bronchial lavage fluid, sputum, saliva, urine, amniotic fluid, lymph fluid, tissue or fine needle biopsy samples, peritoneal fluid, cerebrospinal fluid, nipple aspirates, and includes supernatant from cell lysates, lysed cells, cellular extracts, and nuclear extracts. In some embodiments, the whole blood sample is further processed into serum or plasma samples. In some embodiments, the sample includes blood spotting tests.

The term “subject” or “patient” as used herein, refers to a mammal, preferably a human.

Biomarkers

Biomarkers that may be used include but are not limited to cytokines, chemokines, growth and angiogenic factors, metastasis related molecules, cancer antigens, apoptosis related proteins, proteases, adhesion molecules, cell signaling molecules and hormones. In some embodiments, the biomarkers may be proteins that are circulating in the subject that may be detected from a fluid sample obtained from the subject. In some embodiments, the fluid sample may be serum or plasma. In some embodiments, the biomarker panel may include biomarkers associated with angiogenesis. In some embodiments, one or more biomarkers from a panel of biomarkers may be used.

In some embodiments, the biomarker panel may include 1, 2, 3, 4, 5, 6, 7, 8, 9, 10, 11, 12, 13, 14, 15, 16 or 17 biomarkers. In some embodiments, the biomarker panel may include ten or fewer biomarkers. In yet other embodiments, the biomarker panel may include 1, 2, 3, 4, 5, 6, 7 or 8 biomarkers. In some embodiments, the biomarker panel may be optimized from a candidate pool of biomarkers. By way of non-limiting example, the biomarker panel may be configured for determining whether a subject has a specific disease. In some embodiments, the biomarker panel may be configured to determine whether the subject has lung cancer. In some embodiments, the biomarker panel may be configured for risk stratifying indeterminate nodules found during a low-dose CT-based lung cancer screening. In some embodiments, the biomarker panel may be configured to distinguish between localized disease and locoregional progression. In some embodiments, the biomarker panel may be configured to detect early metastatic disease progression of lung cancer. In some embodiments, the biomarker panel may be configured to determine the likelihood of disease recurrence and outcome. In some embodiments the biomarker panel may be configured to distinguish local metastasis and distant metastasis.

In some embodiments, the biomarker panel may be selected using a reference profile that can be made in conjunction with a statistical algorithm used with a computer to implement the statistical algorithm to sort the subject into a group. In some embodiments, the statistical algorithm is a learning statistical classifier system. The learning statistical classifier system can be selected from the following list of non-limiting examples, including Random Forest (RF), Classification and Regression Tree (CART), boosted tree, neural network (NN), support vector machine (SVM), general chi-squared automatic interaction detector model, interactive tree, multiadaptive regression spline, machine learning classifier, and combinations thereof. By way of non-limiting example, exemplary tools for selecting a biomarker panel may be found in WO 2012/054732 and U.S. Provisional Application No. 61/792,710 which are incorporated by reference herein.

In some embodiments, the biomarker panel may include biomarkers associated with angiogenesis. In some embodiments, the biomarker panel may include at least one biomarker from a panel of seventeen angiogenesis biomarkers. The seventeen angiogenesis biomarkers may include the following: Epidermal growth factor (EGF), Angiopoietin-2, granulocyte colony-stimulating factor (G-CSF), bone morphogenic protein 9 (BMP-9), Endoglin, Endothelin-1, Leptin, fibroblast growth factor-1 (FGF-1), FGF-2, Follistatin, interleukin-8 (IL-8), hepatocyte growth factor (HGF), heparin-binding epidermal growth factor (HB-EGF), placental growth factor (PLGF), vascular endothelial growth factor-A (VEGF-A), VEGF-C and VEGF-D. In some embodiments, other biomarkers may be used and may be combined with one or more of the seventeen biomarkers listed above. In some embodiments, the biomarker panel may include one or more of IL-8, HGF, G-CSF, VEGF-D and FGF-2 or combinations thereof and be configured to distinguish benign nodules from stage I adenocarcinoma. In some embodiments, the biomarker panel may include one or more of EGF, HGF, HB-EGF and VEGF-C or combinations thereof and be configured to distinguish stage I from locally advanced disease. In some embodiments, the biomarker panel may include one or more of endoglin, endothelin-1, leptin, HB-EGF, VEGF-C, VEGF-D and FGF-2 or combinations thereof and be configured to determine the likelihood of disease recurrence. In some embodiments, the biomarker panel include one or more of BMP-9, endoglin, it-8, PLGF, VEGF-C, VEGF-D, FGF-1 and FGF-2 or combinations thereof and be configured to distinguish locally advanced disease from stage IV disease.

Biomarker Panel Measurement

Measurement of one or more biomarkers in a biomarker panel generally relates to a quantitative measurement of an expression product, which is typically a protein or polypeptide. In some embodiments, the measurement of a biomarker panel may relate to a quantitative or qualitative measurement of nucleic acids, such as DNA or RNA.

Expression of the biomarkers may be measured using any method known to one skilled in the art. Methods for measuring protein expression include, but are not limited to Western blot, immunoprecipitation, immunohistochemistry, Enzyme-linked immunosorbent assay (ELISA), Radio Immuno Assay (RIA), radioreceptor assay, proteomics methods, mass-spec based detection (SRM or MRM) or quantitative immunostaining methods. Methods for measuring nucleic acid expression or levels may be any techniques known to one skilled in the art. Expression levels from the panel of biomarkers are measured in the subject and compared to the levels of the panel of biomarkers obtained from a cohort of subjects described below.

In some embodiments, MILLIPLEX® MAP multiplex assays may be used to determine the expression levels of the panel of biomarkers. (EMD Millipore, Billlerica, Mass.) By way of non-limiting example, the MILLIPLEX® MAP Human Angiogenesis/Growth Factor Panel may be used as described in more detail below. In some embodiments, Luminex-based xMAP® multiplexed immunoassays may be used to determine the expression levels of the panel of biomarkers. (Luminex Corp.; Austin, Tex.) In some embodiments, biomarker concentrations may be calculated based on 7-point standard curves using a five-parametric fit algorithm in xPONENT v4.0.3 (Luminex Corp.)

In some embodiments, a kit may be provided with reagents to measure at least one biomarker. In some embodiments, the kit may be provided with reagents to measures at least two biomarkers in a panel of biomarkers. The panel of biomarkers to be measured with the kit may include one or more biomarkers selected from Epidermal growth factor (EGF), Angiopoietin-2, granulocyte colony-stimulating factor (G-CSF), bone morphogenic protein 9 (BMP-9), Endoglin, Endothelin-1, Leptin, fibroblast growth factor-1 (FGF-1), FGF-2, Follistatin, interleukin-8 (IL-8), hepatocyte growth factor (HGF), heparin-binding epidermal growth factor (HB-EGF), placental growth factor (PLGF), vascular endothelial growth factor-A (VEGF-A), VEGF-C and VEGF-D.

The kit may include reagents to measure a panel of biomarkers that includes two, three, four, five, six, seven, eight or more biomarkers combined together to measure a subject's biomarker panel. The kit may be provided with one or more assays provided together in a kit. By way of non-limiting example, the kit may include reagents to measure the biomarkers in one assay. In some embodiments, the kit may include reagents to measure the biomarkers in more than one assay. In some embodiments the kit may include reagents to distinguish benign nodules from stage I adenocarcinoma using the biomarkers described herein. In some embodiments, the kit may include reagents to distinguish stage I from locally advanced disease using the biomarkers described herein. In some embodiments, kit may include reagent configured to determine the likelihood of disease recurrence using the biomarkers described herein. In some embodiments, the kit may include reagents configured to distinguish locally advanced disease from stage IV disease using biomarkers described herein.

Analysis of Biomarker Panel Measurements

In some embodiments, the methods described herein may be based upon one or more biomarker measurements from the subject compared to a reference cutoff level for each biomarker measured. Reference cutoff levels for a plurality of biomarkers are listed in Tables III-V.

Treatment Stratification

In some embodiments, the analysis of the biomarker panel may be used to determine a treatment regime for the subject. In some embodiments, the measurement of one or more biomarkers in the panel may be used to determine whether to begin a treatment, to continue the same treatment or to modify the treatment regime for a subject. The treatment may be modified by changing the drug administered to the subject or to add an additional drug to the existing drug treatment regime, to change the dosage or other changes. In some embodiments, other types of treatment regimes may be used such as radiation.

In some embodiments, angiogenesis biomarkers associated with several discrete stages in the clinical presentation of lung adenocarcinoma were evaluated, including comparisons to relevant control cases for lung cancer detection. An ANOVA analysis with Tukey and LSD post-hoc tests along with the Mann-Whitney Rank Sum U (two-tailed) test were used to evaluate the individual candidate biomarkers mean concentrations across all progressive stages, as defined in ‘Patient Cohorts’. In some embodiments, 2, 3, 4, 5 or more biomarkers were tested and analyzed together. By way of non-limiting example, 2, 3, 4, 5 or more biomarkers having a p value of less than or equal to 0.05 were measured and used to determine a treatment regime. In some embodiments, 17 biomarkers were tested as described in more detail below. Similarly, a Receiver Operator Characteristics (ROC) analysis was also performed for these same comparisons. All analyses described herein were completed using SPSS v19.0 (SPSS Inc., Chicago, Ill.) with significance defined by a two-tailed p-value of less than or equal to 0.05.

In some embodiments, the association of angiogenesis biomarkers with clinical outcome measures was evaluated, included both progression-free survival (PFS) and overall survival (OS). A Mann-Whitney U test was used to evaluate candidate biomarkers mean concentrations between patients with a favorable PFS and OS. Kaplan-Meier curves were generated for both PFS and OS. All statistical analyses were completed using the R Statistical Package. Significance was defined by a two-tailed p-value of less than or equal to 0.05. In some embodiments, 2, 3, 4, 5 or more biomarkers were tested. In some embodiments, 17 biomarkers were tested as described in more detail below.

Subject Cohorts

Between 2004 and 2011, 260 subjects at Rush University Medical Center (RUMC, Chicago, Ill.) were enrolled and divided into the following cohorts: (a) patients at high-risk for lung cancer and negative LDCT imaging (n=20), (b) patients at high-risk for lung cancer with detected (benign) solitary pulmonary nodules (n=36), (c) patients with stage I lung adenocarcinoma without recurrence (n=45) (d) patients with stage I disease with recurrence (n=30) (e) patients with locally-advanced (N+) disease (n=60) and (f) patients with stage IV disease (n=68). All stage classifications were determined according to the American Joint Committee on Cancer (AJCC) seventh edition criteria and confirmed by pathological evaluation (5, 6). Patients enrolled in our institutions lung cancer-screening program (groups a and b) were all considered “high-risk” for lung cancer based on being at least 50 years old and possessing a minimum of 20 pack year smoking history. All patients in these groups remained cancer-free after 2 year of low-dose CT (LDCT) scan follow-up. All patient data was obtained after informed consent was given by the patient. The study was conducted in absolute compliance with the Institutional Review Board at Rush University Medical Center. Demographic information for these subject groups are contained in Table 1.

TABLE I
Patient Demographics

| | | | Benign | | Locally | |
|---|---|---|---|---|---|---|
| | | Control | Nodule | Stage I | Advanced | Stage IV |
| Gender | Female | 12 | 19 | 45 | 35 | 40 |
| | Male | 8 | 17 | 30 | 25 | 28 |
| Age^{a} | Median | 56.5 | 61 | 69 | 68 | 64.5 |
| | Range | 49-73 | 27-83 | 47-92 | 46-89 | 38-87 |
| Smoking | Median | 30 | 37.5 | 30 | 30 | 20 |
| History^{b} | Range | 8-141 | 7-129 | 0-105 | 0-120 | 0-95 |
| Nodule Size^{c} | Median | | 0.4 | 2.2 | | |
| | Range | | 0.2-1.7 | 0.60-13.9 | | |
| TNM (6^{th} Ed.) | T₁N₀M₀ | | | 35 (19 | | |
| | | | | w/rec.) | | |
| | T₂N₀M₀ | | | 40 (11 | | |
| | | | | w/rec.) | | |
| | T₁₋₂N₁₋₂M₀ | | | | 41 | |
| | T₃₋₄N₁₋₂M₀ | | | | 7 | |
| | TₓN₁₋₂M₀ | | | | 12 | 68 |
| | TₓNₓM₁ | | | | | |

Measurement of Serum Biomarker Concentrations

Peripheral blood was collected from patients enrolled in this study either as ‘pretreatment’ (i.e. prior to anatomical resection or frontline chemotherapy) or associated with our institutional lung cancer screening protocol. Serum was prepared using standard phlebotomy protocols and archived at −80° C. in aliquots; no evaluable specimen subjected to more than two freeze-thaw cycles (7-10). Seventeen biomarkers associated with angiogenesis were evaluated in this study (11). All seventeen biomarkers were part of the MILLIPLEX® MAP Human Angiogenesis/Growth Factor Panel (EMD Millipore, Billerica, Mass.) and included the following assays: Epidermal growth factor (EGF), Angiopoietin-2, granulocyte colony-stimulating factor (G-CSF), bone morphogenic protein 9 (BMP-9), Endoglin, Endothelin-1, Leptin, fibroblast growth factor-1 (FGF-1), FGF-2, Follistatin, interleukin-8 (IL-8), hepatocyte growth factor (HGF), heparin-binding epidermal growth factor (HB-EGF), placental growth factor (PLGF), vascular endothelial growth factor-A (VEGF-A), VEGF-C and VEGF-D. All assays were performed in a blinded fashion with data collected on a Luminex FlexMAP 3D system. All biomarker concentrations calculated based on 7-point standard curves using a five-parametric fit algorithm in xPONENT v4.0.3 (Luminex Corp., Austin, Tex.).

Statistical Methods: Cancer Detection and Progression

One endpoint was to evaluate angiogenesis biomarkers associated with several discrete stages in the clinical presentation of lung adenocarcinoma, including comparisons to relevant control cases for lung cancer detection. An ANOVA analysis with Tukey and LSD post-hoc tests along with the Mann-Whitney Rank Sum U (two-tailed) test were used to evaluate the 17 individual candidate biomarkers mean concentrations across all progressive stages, as defined in ‘Patient Cohorts’. Similarly, a Receiver Operator Characteristics (ROC) analysis was also performed for these same comparisons. All analyses described herein were completed using SPSS v19.0 (SPSS Inc., Chicago, Ill.) with significance defined by a two-tailed p-value of less than or equal to 0.05.

Statistical Methods: Progression-Free Survival and Overall Survival

Another endpoint was to evaluate the association of angiogenesis biomarkers with clinical outcome measures, including both progression-free survival (PFS) and overall survival (OS). A Mann-Whitney U test was used to evaluate candidate biomarkers mean concentrations between patients with a favorable PFS and OS. Kaplan-Meier curves were generated for both PFS and OS. All statistical analyses were completed using the R Statistical Package. Significance was defined by a two-tailed p-value of less than or equal to 0.05.

Results

Clinical Parameter Analysis

A total of 259 cases were enrolled in this study and divided into several discrete cohorts: patients at high-risk for lung cancer and negative LDCT imaging (n=20); patients at high-risk for lung cancer with detected (benign) solitary pulmonary nodules (n=36); patients with stage I lung adenocarcinoma without recurrence (n=45); patients with stage I disease with recurrence (n=30); patients with locally-advanced (N+) disease (n=60); and patients with stage IV disease (n=68). Basic patient demographics are provided for the overall cohort in Table I. A multivariate analysis (both in each group and overall) was then performed to determine if any of the basic clinical parameters were significantly associated with biomarker levels. The multivariate analysis found gender to be associated with serum leptin and VEGF-A levels (p<0.001 and 0.047, respectively), whereas age was significantly associated only with the leptin levels (p=0.01). Smoking history was found to be associated with BMP-9, HB-EGF, and VEGF-A levels (p=0.0028, 0.0094, and 0.0355, respectively).

General Trends in Angiogenesis Biomarker Levels

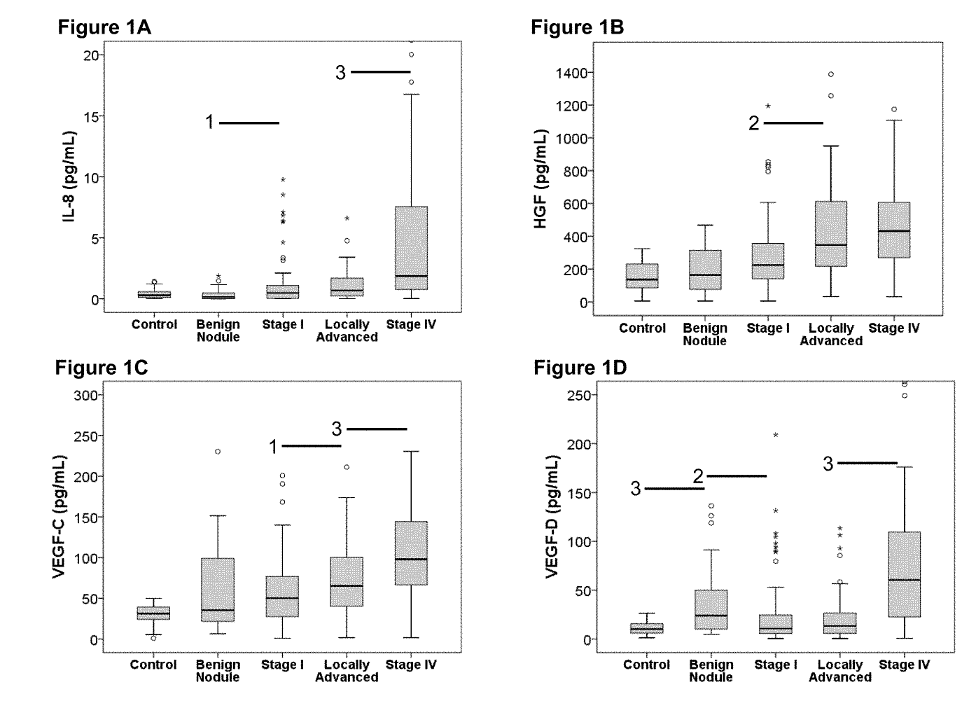

One objective of this study was to evaluate the trends in circulating angiogenesis biomarker levels at several discrete stages in the clinical presentation of lung adenocarcinoma, including comparisons to relevant control cases for lung cancer detection. The observed levels broken down by groups are presented in Table II. Thirteen of the seventeen biomarkers were found to have significance in detecting at least one phase of progression, with ‘box and whisker’ plots of the most compelling biomarkers (IL-8, HGF, VEGF-C, and VEGF-D) shown in FIG. 1. Specific comparisons relevant to clinical decision making are focused on in the sections below.

Indeterminate Solitary Nodules Detected Via Low-Dose CT Scans

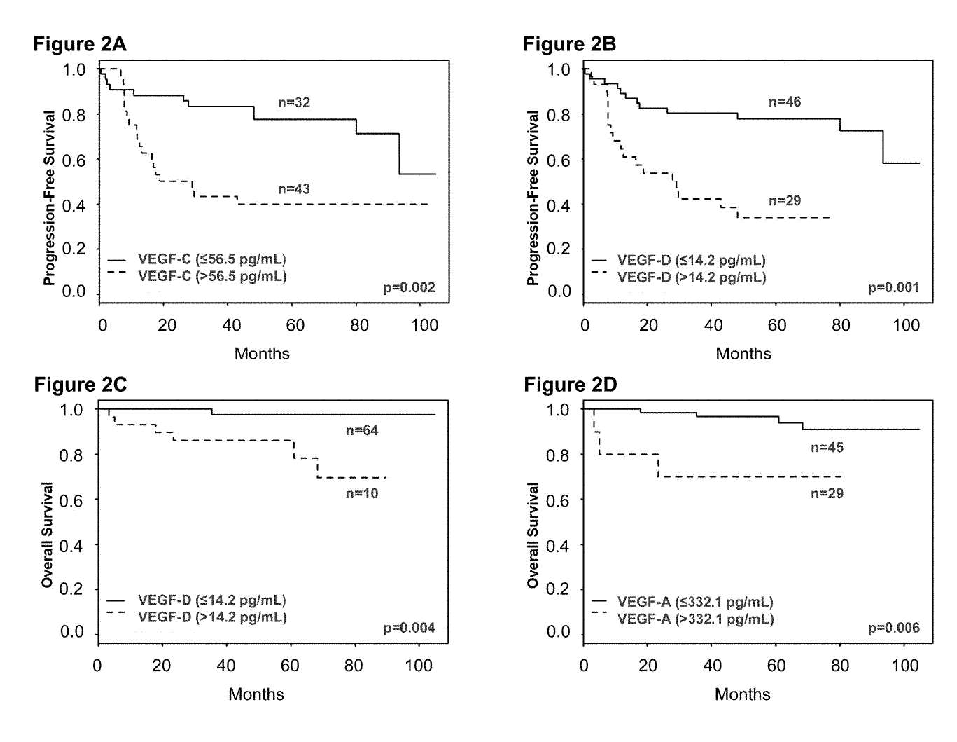

The development of a companion diagnostic for LDCT-based screening programs that can help stratify patients with indeterminate solitary pulmonary nodules into more timely treatment plans is of significant interest. In this regard, we reasoned that angiogenesis would be less pronounced and at an earlier phase of this process in the proliferative adenocarcinoma masses versus a typically slower growing benign nodule. To test this idea, angiogenesis biomarkers were evaluated against the serum from 36 cases of individuals that were part of a lung cancer screening study at Rush with positive CT results that were stable for 2 years in clinical follow-up and compared to the results with 75 cases with pathologically-confirmed stage I (T₁₋₂N₀M₀) disease. From this study, G-CSF, FGF-2, IL-8, and VEGF-D were found to all have significant Mann-Whitney Rank Sum (two-tailed) p-values, as shown in Table II and partially-represented in FIGS. 2A and 2B). A ROC analysis of these biomarkers revealed ‘area-under-the curve’ values that were 0.733, 0.654, 0.644, and 0.680, respectively (data not shown).

Detection of Early Metastatic Progression

The serum levels of angiogenesis biomarkers were compared in patients with either stage I disease (T₁₋₂N₀M₀; n=75) or locally-advanced (T₁₋₄N₁₋₂M₀; n=60) adenocarcinoma in pursuit of biomarkers of early metastatic progression. From this analysis, the serum levels of four angiogenesis biomarkers were found to be significantly altered between the two cohorts: EGF, HGF, VEGF-C and HB-EGF. Observed levels of all biomarkers are shown in Table II. All four biomarkers were found to be elevated in patients with node positive lung cancer. A ROC analysis of these biomarkers revealed ‘area-under-the curve’ values that were 0.601, 0.652, 0.606, 0.604, respectively (data shown in Table V).

Prognostication of Disease Recurrence and Outcome in the Stage I Cohort

An examination within the stage I cohort for associations of angiogenesis biomarkers and outcome parameters was explored. Within this cohort the median time to recurrence was 53 weeks and median survival was 4.57 years, versus 5.78 years for those with no documented incidence of recurrence. In terms of progression-free survival (PFS), low levels of angiopoietin-2, BMP-9, endoglin, endothelin-1, FGF-1, FGF 2, HB-EGF, PLGF, VEGF-C, and VEGF-D were found to have the best outcomes and possess significant (p≤0.05) log-rank p-values. For overall survival (OS), angiopoietin-2, endoglin, follistatin, PLGF, VEGF-A and VEGF-D were the biomarkers with significance. All log-rank values are shown in Table III with relevant cutoff values and number of patients in each group specified.

Progression from Locally-Advanced to Disseminated Disease

Finally, biomarker levels of the cohort of locally-advanced adenocarcinoma (n=60) were compared with the cohort of stage IV adenocarcinomas (n=68) in an attempt to distinguish biomarker profiles for local metastasis and distant metastasis. All sera were collected pre-treatment using identical protocols, despite the differences in clinical management strategies. The analysis for these data was performed in two manners: a comparison of median levels with disease status and a prognostic comparison using a log-rank analysis. Significantly higher levels (p≤0.05) of BMP-9, endoglin, FGF-1, FGF-2, IL-8, PLGF, VEGF-C, and VEGF-D were observed in the stage IV cohort, as presented in Table II and shown in FIG. 1.

Associations of biomarker levels with clinical outcomes in the locally-advanced cohort were weak (data not shown), with only endothelin-1 found to be significant (p≤0.05) when cutoff values were optimized to the stage IV cases; however, both endothelin-1 and angiopoietin-2 were found to be significant (p≤0.05) when optimized independently. High levels of the following angiogenesis biomarkers significantly (p≤0.05) associated with a favorable outcome in terms PFS: EGF, endothelin-1, and leptin; whereas low levels of FGF-1 and VEGF-D were more beneficial. Similarly, high levels of EGF, endothelin-1, BMP-9, leptin, and VEGF-A were associated with a favorable OS; whereas low levels of angiopoietin-2 were correlated with improved OS. Again, if cutoff values for the locally advanced cohort was obtained independent of the stage IV cases G-CSF and IL-8 were also statistically significant (p=0.031 and 0.018, respectively; data not shown). The analysis of the stage IV cohort for association of biomarker levels with PFS and OS using log-rank methods are provided in Table IV.

A systematic evaluation of a range of pro-angiogenic biomarkers in the context of assigning clinical significance to CT-detected indeterminate solitary pulmonary nodules has been described herein.

The practice of the present invention will employ, unless otherwise indicated, conventional methods for measuring the level of the biomarker within the skill of the art. The techniques may include, but are not limited to, molecular biology and immunology. Such techniques are explained fully in the literature. See, e.g., Sambrook, et al. Molecular Cloning: A Laboratory Manual (Current Edition, Cold Spring Harbor Laboratory Press, Cold Spring Harbor, N.Y.); Current Protocols in Molecular Biology (Eds. A Ausubel et al., NY: John Wiley & Sons, Current Edition); DNA Cloning: A Practical Approach, vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition).

The above Figures and disclosure are intended to be illustrative and not exhaustive. This description will suggest many variations and alternatives to one of ordinary skill in the art. All such variations and alternatives are intended to be encompassed within the scope of the attached claims. Those familiar with the art may recognize other equivalents to the specific embodiments described herein which equivalents are also intended to be encompassed by the attached claims.

TABLE II
Observed levels of angiogenesis biomarkers in sera from our 5 cohorts.

| | Control | | Control vs. | Benign Nodule | | Benign | Stage I | |
|---|---|---|---|---|---|---|---|---|
| | Median | | Benign | Median | | Nodule | Median | |
| | (pg/mL) | Range | Nodule | (pg/mL) | Range | vs. Stage I | (pg/mL) | Range |
| EGF | 22.24 | 0.5-235.1 | 0.837 | 22.28 | 1.9-134.0 | 0.992 | 20.30 | 0.5-320.8 |
| Angiopoietin-2 | 1277 | 653-2410 | 0.824 | 1271 | 287-2811 | 0.533 | 1244 | 407-4023 |
| G-CSF | 32.5 | 7.0-235.5 | 0.771 | 40.4 | 0.4-355.3 | <0.001 | 6.99 | 0.4-79.1 |
| BMP-9 | 146.6 | 54-265.9 | 0.986 | 145.7 | 36-419.1 | 0.176 | 128.4 | 1-368.1 |
| Endoglin | 231.8 | 26-512.4 | 0.010 | 458.6 | 26-1347 | 0.691 | 500.0 | 94-1530 |
| Endothelin-1 | 0.24 | 0.2-16.9 | 0.026 | 3.60 | 0.2-66.1 | 0.224 | 2.91 | 0.2-68.1 |
| Leptin* | 5.85 | 0.7-42.7 | 0.161 | 9.97 | 0.5-135.0 | 0.865 | 11.05 | 0.9-84.4 |
| FGF-1 | 2.51 | 2.5-27.6 | 0.041 | 2.51 | 0.2-746.4 | 0.764 | 2.51 | 0.2-555.1 |
| FGF-2 | 32.6 | 19.2-117.5 | 0.322 | 43.8 | 19.2-357.3 | 0.007 | 20.3 | 19.2-102.2 |
| Follistatin | 377.7 | 164.2-879.9 | 0.009 | 521.2 | 90.7-2138 | 0.279 | 618.8 | 21.1-2052 |
| IL-S | 0.26 | 0.01-1.41 | 0.226 | 0.15 | 0.01-1.88 | 0.014 | 0.48 | 0.01-73.8 |
| HGF | 136.8 | 5.3-324.0 | 0.378 | 164.3 | 5.3-468.0 | 0.062 | 224.5 | 5.3-1194 |
| HB-EGF | 59.7 | 28.0-109.8 | 0.281 | 72.5 | 3.5-171.7 | 0.219 | 81.3 | 21.9-236.8 |
| PLGF | 1.59 | 0.01-7.68 | 0.278 | 1.75 | 0.01-74.27 | 0.564 | 2.45 | 0.01-16.41 |
| VEGF-A | 54.6 | 5.0-186.3 | 0.146 | 109.6 | 5.0-779.1 | 0.106 | 162.2 | 4.5-1145.2 |
| VEGF-C | 31.2 | 1.0-49.9 | 0.171 | 35.5 | 6.5-230.4 | 0.450 | 51.3 | 1.0-200.9 |
| VEGF-D | 11.0 | 1.3-26.4 | 0.001 | 24.4 | 4.8-136.3 | 0.002 | 10.7 | 0.5-209.1 |

| | Stage I vs. | Locally-Advanced | | Locally | Stage IV | |
|---|---|---|---|---|---|---|
| | Locally | Median | | Advanced | Median | |
| | Advanced | (pg/mL) | Range | vs. Stage IV | (pg/mL) | Range |
| EGF | 0.044 | 35.06 | 0.5-978.6 | 0.179 | 18.05 | 1-541.0 |
| Angiopoietin-2 | 0.066 | 1610 | 458-7583 | 0.875 | 1440 | 510-14251 |
| G-CSF | 0.104 | 8.76 | 0.4-1278 | 0.064 | 2.80 | 0.4-182.5 |
| BMP-9 | 0.193 | 111.6 | 4-501.6 | 0.019 | 154.2 | 1-558.8 |
| Endoglin | 0.072 | 562.2 | 26-1218 | 0.005 | 799.9 | 71-2077 |
| Endothelin-1 | 0.171 | 3.64 | 0.2-101.7 | 0.088 | 3.64 | 0.2-227.0 |
| Leptin* | 0.828 | 9.96 | 0.3-103.5 | 0.650 | 9.38 | 1.8-71.8 |
| FGF-1 | 0.514 | 3.56 | 0.2-663.3 | <0.001 | 11.1 | 2.4-2774.2 |
| FGF-2 | 0.139 | 29.8 | 19.2-233.6 | 0.002 | 43.8 | 19.2-313.4 |
| Follistatin | 0.319 | 621.9 | 79.9-2954 | 0.057 | 804.24 | 180.9-7647 |
| IL-S | 0.255 | 0.68 | 0.01-27151 | <0.001 | 1.87 | 0.02-417 |
| HGF | 0.002 | 346.8 | 32.2-1388 | 0.170 | 431.8 | 31.2-4268.8 |
| HB-EGF | 0.035 | 96.1 | 20.8-237.0 | 0.453 | 103.6 | 21.0-406.0 |
| PLGF | 0.204 | 3.31 | 0.01-18.93 | <0.001 | 8.94 | 0.01-74.08 |
| VEGF-A | 0.089 | 189.8 | 4.5-2089.3 | 0.754 | 203.5 | 4.5-2631.7 |
| VEGF-C | 0.038 | 65.3 | 1.63-211.2 | 0.002 | 98.0 | 1.6-1362.3 |
| VEGF-D | 0.640 | 13.4 | 0.5-273.8 | <0.001 | 60.4 | 0.7-691.0 |

*Values provided are in ng/mL

TABLE III
Association of Angiogenesis Biomarkers with Clinical Outcomes in Stage I Lung Adenocarcinoma.
Provided are the values calculated for the progression-free survival (PFS) and overall
survival (OS) of the stage I cohort with respect to the serum levels of the angiogenesis
biomarkers tested. Cutoff values are provided (optimized for greatest significance
in each outcome measure) as well as the number of cases below and equal to the cutoff
threshold value and the median survival times, in weeks, below (‘median low’)
and above (‘median high’) the indicated threshold values.

| | PFS | | | | | OS | | | | |
|---|---|---|---|---|---|---|---|---|---|---|
| | Cutoff | | Median- | Median | | Cutoff | | Median- | Median | |
| | (pg/mL) | n≤ | Low² | High² | p | (pg/mL) | n≤ | Low² | High² | p |
| EGF | 97.1 | 66 | 93.4 | 3 | 0.107 | 74.5 | 64 | ³ | ³ | 0.284 |
| Angiopoietin-2 | 1967.9 | 60 | ³ | 43 | 0.018 | 2031.8 | 60 | ³ | ³ | 0.006 |
| G-CsF | 2.19 | 30 | 48.1 | 93.4 | 0.105 | 7.0 | 38 | ³ | ³ | 0.225 |
| BMP-9 | 191.9 | 60 | ³ | 29.6 | 0.023 | 222.8 | 64 | ³ | ³ | 0.171 |
| Endoglin | 882.9 | 67 | 93.4 | 23.2 | 0.034 | 646.3 | 51 | ³ | ³ | 0.012 |
| Endothelin-1 | 2.91 | 38 | ³ | 43 | 0.019 | 22.7 | 66 | ³ | ³ | 0.375 |
| Leptin ¹ | 15.7 | 45 | ³ | 80.1 | 0.082 | 6.4 | 26 | ³ | ³ | 0.247 |
| FGF-1 | 16.5 | 62 | ³ | 16.4 | 0.009 | 2.5 | 41 | ³ | ³ | 0.442 |
| Follistatin | 874.2 | 60 | 93.4 | 48.1 | 0.193 | 558.1 | 29 | ³ | ³ | 0.034 |
| IL-8 | 0.18 | 24 | ³ | 80.1 | 0.075 | 0.18 | 24 | ³ | ³ | 0.064 |
| HGF | 380.0 | 58 | 93.4 | 27.8 | 0.109 | 139.2 | 17 | ³ | ³ | 0.127 |
| HB-EGF | 83.6 | 39 | ³ | 48.1 | 0.032 | 48.1 | 14 | ³ | ³ | 0.208 |
| PLGF | 2.17 | 34 | ³ | 48.1 | 0.009 | 3.9 | 45 | ³ | ³ | 0.047 |
| VEGF-C | 56.5 | 43 | ³ | 23.9 | 0.002 | 33.5 | 22 | ³ | ³ | 0.077 |
| VEGF-D | 14.2 | 46 | ³ | 27.8 | <0.001 | 14.3 | 45 | ³ | ³ | 0.004 |
| FGF-2 | 41.1 | 60 | ³ | 43 | 0.014 | 41.1 | 59 | ³ | ³ | 0.116 |
| VEGF-A | 131.8 | 32 | 80.1 | ³ | 0.188 | 332.1 | 64 | ³ | ³ | 0.006 |

¹ Values provided are in ng/mL;
²Value (in weeks) where survival falls below 0.5;
³ Curves didn't cross 0.5 value.

TABLE IV
Association of Angiogenesis Biomarkers with Clinical Outcome in Stage IV Adenocarcinoma.
Provided are the values calculated for the progression-free survival (PFS) and overall
survival (OS) of the stage IV cohort with respect to the serum levels of the angiogenesis
biomarkers tested. Cutoff values are provided (optimized for greatest significance
in each outcome measure) as well as the number of cases below and equal to the cutoff
threshold value and the median survival times, in weeks, below (‘median low’)
and above (‘median high’) the indicated threshold values.

| | PFS | | | | | OS | | | | |
|---|---|---|---|---|---|---|---|---|---|---|
| | Cutoff | | Median- | Median | | Cutoff | | Median- | Median | |
| | (pg/mL) | n≤ | Low² | High² | p | (pg/mL) | n≤ | Low² | High² | p |
| EGF | 7.23 | 22 | 3.2 | 5.9 | 0.023 | 7.23 | 22 | 6.8 | 13.2 | 0.038 |
| Angiopoietin- | 2396.1 | 55 | 5.2 | 2.9 | 0.218 | 2396.1 | 55 | 14.2 | 9.1 | 0.022 |
| G-CsF | 0.92 | 58 | 5.3 | 2.8 | 0.130 | 0.92 | 17 | 17.5 | 9.4 | 0.449 |
| BMP-9 | 123.4 | 21 | 2.0 | 6.1 | 0.068 | 123.4 | 26 | 8.0 | 17.9 | 0.018 |
| Endoglin | 916.1 | 57 | 5.7 | 2.4 | 0.066 | 916.1 | 43 | 13.3 | 6.1 | 0.208 |
| Endothelin-1 | 3.64 | 39 | 3.4 | 6.8 | 0.004 | 3.64 | 39 | 7.9 | 17.9 | 0.019 |
| Leptin ¹ | 8.11 | 11 | 2.9 | 5.4 | 0.018 | 8.11 | 28 | 7.0 | 17.5 | 0.024 |
| FGF-1 | 3.56 | 38 | 5.5 | 3.1 | 0.039 | 3.56 | 24 | 11.6 | 11.5 | 0.270 |
| Follistatin | 1078.8 | 9 | 2.4 | 5.4 | 0.117 | 1078.8 | 47 | 9.4 | 21.7 | 0.137 |
| IL-8 | 11.7 | 35 | 6.1 | 2.9 | 0.105 | 11.7 | 56 | 11.6 | 10.7 | 0.570 |
| HGF | 608.0 | 49 | 6.0 | 2.9 | 0.099 | 608.0 | 51 | 13.0 | 4.4 | 0.075 |
| HB-EGF | 133.1 | 29 | 4.4 | 5.4 | 0.146 | 133.1 | 49 | 9.2 | 18.3 | 0.065 |
| PLGF | 1.00 | 46 | 6.0 | 3.1 | 0.099 | 1.00 | 10 | 9.5 | 12.4 | 0.137 |
| VEGF-C | 91.2 | 11 | 4.3 | 5.2 | 0.249 | 91.2 | 31 | 9.4 | 14.2 | 0.136 |
| VEGF-D | 41.0 | 48 | 6.1 | 2.8 | 0.031 | 41.0 | 26 | 17.7 | 9.1 | 0.069 |
| FGF-2 | 25.9 | 27 | 4.4 | 6.8 | 0.071 | 25.9 | 23 | 8.1 | 14.4 | 0.069 |
| VEGF-A | 120.6 | 20 | 4.6 | 5.3 | 0.064 | 120.6 | 20 | 9.3 | 13.0 | 0.047 |

¹ Values provided are in ng/mL;
²Value (in weeks) where survival falls below 0.5

TABLE V
Benign Nodules vs. Stage I Adenocarcinoma

| Biomarker | AUC | Sensitivity | Specificity | Cutoff conc.¹ |
|---|---|---|---|---|
| IL-8 | 0.644 | 0.707 | 0.417 | 0.084 |
| HGF | 0.610 | 0.720 | 0.472 | 152.4 |
| G-CSF | 0.733 | 0.722 | 0.667 | 10.2 |
| VEGF-D | 0.680 | 0.750 | 0.480 | 10.5 |
| FGF-2 | 0.654 | 0.722 | 0.653 | 28.1 |

Stage I vs. Locally-Advanced Disease

| | | | | |
|---|---|---|---|---|
| EGF | 0.601 | 0.700 | 0.530 | 21.2 |
| HGF | 0.652 | 0.717 | 0.573 | 239.1 |
| HB-EGF | 0.606 | 0.707 | 0.383 | 70.2 |
| VEGF-C | 0.604 | 0.633 | 0.520 | 52.3 |

Stage I - Disease Recurrence

| | | | | |
|---|---|---|---|---|
| Endoglin | 0.616 | 0.633 | 0.533 | 490.9 |
| Endothelin-1 | 0.614 | 0.667 | 0.622 | 3.26 |
| Leptin² | 0.601 | 0.600 | 0.600 | 11.6 |
| HB-EGF | 0.600 | 0.600 | 0.630 | 84.4 |
| VEGF-C | 0.628 | 0.667 | 0.711 | 54.3 |
| VEGF-D | 0.672 | 0.733 | 0.622 | 10.5 |
| FGF-2 | 0.601 | 0.667 | 0.622 | 20.3 |

Locally-Advanced vs. Stage IV Disease

| | | | | |
|---|---|---|---|---|
| BMP-9 | 0.621 | 0.706 | 0.450 | 100.1 |
| Endoglin | 0.644 | 0.603 | 0.617 | 675.1 |
| IL-8 | 0.720 | 0.706 | 0.650 | 0.89 |
| PLGF | 0.683 | 0.721 | 0.550 | 3.9 |
| VEGF-C | 0.662 | 0.750 | 0.533 | 70.2 |
| VEGF-D | 0.763 | 0.779 | 0.617 | 18.0 |
| FGF-1 | 0.679 | 0.647 | 0.550 | 3.78 |
| FGF-2 | 0.654 | 0.603 | 0.617 | 37.0 |

¹All values are provided in pg/mL, except ²which is in ng/mL.

### REFERENCES

1. Hanahan D, Weinberg R A. Hallmarks of cancer: the next generation. Cell 2011; 144: 646-74.
2. Hanahan D, Folkman J. Patterns and emerging mechanisms of the angiogenic switch during tumorigenesis. Cell 1996; 86: 353-64.
3. Raica M, Cimpean A M, Ribatti D. Angiogenesis in pre-malignant conditions. Eur J Cancer 2009; 45: 1924-34.
4. Salgia R. Prognostic significance of angiogenesis and angiogenic growth factors in nonsmall cell lung cancer. Cancer 2011; 117: 3889-99.
5. Goldstraw P, Crowley J, Chansky K, et al. The IASLC Lung Cancer Staging Project: proposals for the revision of the TNM stage groupings in the forthcoming (seventh) edition of the TNM Classification of malignant tumours. J Thorac Oncol 2007; 2: 706-14.
6. Groome P A, Bolejack V, Crowley J J, et al. The IASLC Lung Cancer Staging Project: validation of the proposals for revision of the T, N, and M descriptors and consequent stage groupings in the forthcoming (seventh) edition of the TNM classification of malignant tumours. J Thorac Oncol 2007; 2: 694-705.
7. Borgia J A, Basu S, Faber L P, et al. Establishment of a multi-analyte serum biomarker panel to identify lymph node metastases in non-small cell lung cancer. J Thorac Oncol 2009; 4: 338-47.
8. Farlow E C, Patel K, Basu S, et al. Development of a multiplexed tumor-associated autoantibody-based blood test for the detection of non-small cell lung cancer. Clin Cancer Res 2010; 16: 3452-62.
9. Patel K, Farlow E C, Kim A W, et al. Enhancement of a multianalyte serum biomarker panel to identify lymph node metastases in non-small cell lung cancer with circulating autoantibody biomarkers. Int J Cancer 2010; 129: 133-42.
10. Shersher D D, Vercillo M S, Fhied C, et al. Biomarkers of the Insulin-Like Growth Factor Pathway Predict Progression and Outcome in Lung Cancer. Ann Thorac Surg 2011.
11. Rinewalt D, Shersher D D, Daly S, et al. Development of a serum biomarker panel predicting recurrence in stage I non-small cell lung cancer patients. J Thorac Cardiovasc Surg 2012; 144: 1344-50; discussion 50-1.
12. Yang Y, Sun M, Wang L, Jiao B. HIFs, angiogenesis, and cancer. J Cell Biochem 2013; 114: 967-74.
13. Seftor R E, Hess A R, Seftor E A, et al. Tumor cell vasculogenic mimicry: from controversy to therapeutic promise. Am J Pathol 2012; 181: 1115-25.
14. Daly S, Rinewalt D, Fhied C, et al. Development and validation of a plasma biomarker panel for discerning clinical significance of indeterminate pulmonary nodules. J Thorac Oncol 2013; 8: 31-6.
15. Baj-Krzyworzeka M, Weglarczyk K, Mytar B, Szatanek R, Baran J, Zembala M. Tumour-derived microvesicles contain interleukin-8 and modulate production of chemokines by human monocytes. Anticancer Res 2011; 31: 1329-35.
16. Joimel U, Gest C, Soria J, et al. Stimulation of angiogenesis resulting from cooperation between macrophages and MDA-MB-231 breast cancer cells: proposed molecular mechanism and effect of tetrathiomolybdate. BMC Cancer 2010; 10: 375.
17. Ribatti D. Mast cells and macrophages exert beneficial and detrimental effects on tumor progression and angiogenesis. Immunol Lett 2013; 152: 83-8.
18. Semkova I, Muether P S, Kuebbeler M, Meyer K L, Kociok N, Joussen A M. Recruitment of blood-derived inflammatory cells mediated via tumor necrosis factor-alpha receptor 1 b exacerbates choroidal neovascularization. Invest Ophthalmol Vis Sci 2011; 52: 6101-8.
19. de Nigris F, Schiano C, Infante T, Napoli C. CXCR4 inhibitors: tumor vasculature and therapeutic challenges. Recent Pat Anticancer Drug Discov 2012; 7: 251-64.
20. Sun X, Charbonneau C, Wei L, Yang W, Chen Q, Terek R M. CXCR4-targeted therapy inhibits VEGF expression and chondrosarcoma angiogenesis and metastasis. Mol Cancer Ther 2013; 12: 1163-70.
21. Tu Z L, Yu G W, Hu Z R, Ni Y M, Ye D S. [Value of serum-vascular endothelial growth factor in the differential diagnosis of solitary pulmonary nodule]. Zhonghua Zhong Liu Za Zhi 2003; 25: 154-6.
22. Bai R J, Cheng X G, Qu H, Shen B Z, Han M J, Wu Z H. Solitary pulmonary nodules: comparison of multi-slice computed tomography perfusion study with vascular endothelial growth factor and microvessel density. Chin Med J (Engl) 2009; 122: 541-7.
23. Ma S H, Le H B, Jia B H, et al. Peripheral pulmonary nodules: relationship between multi-slice spiral CT perfusion imaging and tumor angiogenesis and VEGF expression. BMC Cancer 2008; 8: 186.
24. Lambrechts D, Lenz H J, de Haas S, Carmeliet P, Scherer S J. Markers of response for the antiangiogenic agent bevacizumab. J Clin Oncol 2013; 31: 1219-30.
25. Soria J C, Mauguen A, Reck M, et al. Systematic review and meta-analysis of randomised, phase II/III trials adding bevacizumab to platinum-based chemotherapy as first-line treatment in patients with advanced non-small-cell lung cancer. Ann Oncol 2013; 24: 20-30.

1. A method for determining lung cancer disease progression or for pathological stratification of solitary indeterminate nodules detected during lung cancer screening or for determining the likelihood of disease progression in a subject, the method comprising:
obtaining a biological sample from the subject;
assaying a level in the biological sample of a biomarker in a panel of biomarkers, the panel comprising at least one biomarker selected from the group consisting of Epidermal growth factor (EGF), Angiopoietin-2, granulocyte colony-stimulating factor (G-CSF), bone morphogenic protein 9 (BMP-9), Endoglin, Endothelin-1, Leptin, fibroblast growth factor-1 (FGF-1), FGF-2, Follistatin, interleukin-8 (IL-8), hepatocyte growth factor (HGF), heparin-binding epidermal growth factor (HB-EGF), placental growth factor (PLGF), vascular endothelial growth factor-A (VEGF-A), VEGF-C and VEGF-D;
comparing the biomarker level in the subject's sample to a cutoff value for each biomarker measured for the panel of biomarkers; and
determining whether the biomarker level is above or below the cutoff value.

2. The method according to claim 1, comprising determining the level of at least two biomarkers of the panel of biomarkers.

3. The method according to claim 1, comprising determining the level of the at least one biomarker of the panel of biomarkers wherein the at least one biomarker is selected from the group consisting of IL-8, HGF, G-CSF, VEGF-D and FGF-2.

4. The method according to claim 1, comprising determining the level of biomarkers in the panel of biomarkers wherein the panel comprises IL-8, HGF, G-CSF, VEGF-D and FGF-2

5. The method according to claim 3, wherein the panel of biomarkers distinguishes benign nodules from stage I adenocarcinoma.

6. The method according to claim 1, comprising determining the level of the at least one biomarker of the panel of biomarkers wherein the at least one biomarker is selected from the group consisting of EGF, HGF, HB-EGF and VEGF-C.

7. The method according to claim 1, comprising determining the level of biomarkers in the panel of biomarkers wherein the panel comprises EGF, HGF, HB-EGF and VEGF-C

8. The method according to claim 6, wherein the panel of biomarkers distinguishes stage I disease from locally advanced disease.

9. The method according to claim 1, comprising determining the level of the at least one biomarker of the panel of biomarkers wherein the at least one biomarker is selected from the group consisting of endoglin, endothelin-1, leptin, HB-EGF, VEGF-C, VEGF-D and FGF-2.

10. The method according to claim 1, comprising determining the level of biomarkers in the panel of biomarkers wherein the panel comprises endoglin, endothelin-1, leptin, HB-EGF, VEGF-C, VEGF-D and FGF-2.

11. The method according to claim 10, wherein the panel of biomarkers identifies subjects likely to have disease recurrence.

12. The method according to claim 1, comprising determining the level of the at least one biomarker of the panel of biomarkers wherein the at least one biomarker is selected from the group consisting of BMP-9, endoglin, IL-8, PLGF, VEGF-C, VEGF-D, FGF-1 and FGF-2.

13. The method according to claim 1, comprising determining the level of biomarkers in the panel of biomarkers wherein the panel comprises BMP-9, endoglin, IL-8, PLGF, VEGF-C, VEGF-D, FGF-1 and FGF-2.

14. The method according to claim 12, wherein the panel of biomarkers distinguishes locally advanced disease from stage IV disease.

15. The method according to claim 1, wherein the biological sample comprises plasma sample or serum sample.

16. The method according to claim 1, further comprising placing the subject in a treatment regime based on the biomarker level relative to the cutoff value.

17. A kit for performing the measurement of the panel of biomarkers of the subject in claim 1, wherein the kit comprises reagents for measuring at least one of the panel of biomarkers and the cutoff values listed in Tables III, IV or V.
